(19) United States
(12) Patent Application Publication
(10) Pub. No.: US 2025/0208081 A1
(43) Pub. Date: Jun. 26, 2025

(54) MEASURING DEVICE COMPRISING A FITTING WITH A FLOW CHANNEL AND A METHOD FOR ITS OPERATION

(71) Applicant: Endress+Hauser Conducta GmbH+Co. KG, Gerlingen (DE)

(72) Inventors: Lukas Graf, Mutlangen (DE); Holger Lippert, Leisnig (DE); Johannes Kienle, Gärtringen (DE)

(21) Appl. No.: 18/982,040

(22) Filed: Dec. 16, 2024

(30) Foreign Application Priority Data
Dec. 20, 2023 (DE) 10 2023 135 967.3

(51) Int. Cl. G01N 27/27; G01N 21/27; G01N 21/53; G01N 27/416; G01N 27/49; G01N 33/18

(52) U.S. Cl. CPC G01N 27/27; G01N 21/274; G01N 21/532; G01N 27/4163; G01N 27/4167; G01N 27/49; G01N 33/18; G01N 2201/023; G01N 2201/0245; G01N 2201/127

(57) ABSTRACT

A modular flow measuring device comprises a controller, a third module with an optical sensor connected to the controller, and a fourth module with an amperometric sensor connected to the controller.

### CROSS-REFERENCE TO RELATED APPLICATION

The present application is related to and claims the priority benefit of German Patent Application No. 10 2023 135 967.3, filed on Dec. 20, 2023, the entire contents of which are incorporated herein by reference.

### TECHNICAL FIELD

The present disclosure relates to a module for a modular flow device, to a modular flow measuring device, and to a method for its operation.

### BACKGROUND

An optical sensor or an amperometric sensor can be used to determine a concentration of a dissolved species in a measuring medium. However, due to legal regulations, the measured values of the amperometric sensor must be traced back to the standardized measured values of a chemical reaction which results in coloration. This coloration can then be evaluated using an optical system. This measured value can then be compared or calibrated by the amperometric sensor. Measurement using an optical sensor has the following disadvantages. It is cross-sensitive in the case of changing environmental influences, to other species in the solution beyond the actual species to be determined, and requires chemicals for color indicators. A handheld device is typically used to calibrate or compare the amperometric sensor at a measuring point. This involves taking a sample, an optical measurement by the handheld device using a cuvette, and a comparison with a measurement from the amperometric sensor.

As an alternative to handheld devices, online or inline measuring devices are also known, which carry out a measurement in the flow of the measuring medium. In this case, dosing can be carried out using the color indicator, and the colored measuring medium can be conveyed through a downstream flow cell with a photometer or a colorimeter. It is problematic if the color indicator causes disposal problems, e.g., due to toxicity or environmental damage. The measuring device is a comparatively heavy laboratory device. The consumption of chemicals is also comparatively high. In contrast, the amperometric measurement does not involve any chemical consumption.

Furthermore, optical sensors for concentration determination are known per se. A typical example of this is the determination of the chlorine content according to the DPD measurement method. In part, these must be carried out daily in swimming pools. In smaller systems, a handheld device can be used for this purpose. In the handheld variant, the measuring medium is filled into a cuvette, mixed with N,N-diethyl-1,4-phenylenediamine and then measured. The measurement can be carried out colorimetrically or photometrically. Furthermore, online measuring devices are known for larger swimming pools or industrial applications, which, unlike a handheld device, are correspondingly heavy and typically are not transported to different measuring locations. Rather, the online measuring device is installed near a measuring location, the measuring device having a fluid connection to the measuring location. Thus, the measuring device is “connected to” the measuring location or the sampling location, i.e., online. Sampling is typically done via a pump. The reagents, e.g., the DPD, are added to the measuring medium by a dosing system integrated in the measuring device. In this case, due to the more frequent measurements, there is typically a higher consumption of chemicals than with a handheld device. What both methods have in common is that they measure discontinuously and therefore cannot be used for an automatic control loop.

Turbidity sensors for determining the turbidity of a liquid are also known. They are mostly used in the so-called scattered light method. Turbidity can be influenced by different flow effects, settling effects and the like. At the same time, the turbidity influences the concentration measurement. It is therefore optimal if the turbidity measurement and the concentration determination are carried out as temporally close together as possible or at the same time. Furthermore, in order to reduce measurement inaccuracies, it is advantageous if the two measurements are carried out as far as possible with the same fraction of the measuring medium. Turbidity meters are calibrated using environmentally harmful formazin. If an inline calibration is performed, the formazin would pose a problem when disposing of it and when cleaning the flow cell.

### SUMMARY

The present disclosure therefore starts from the aforementioned prior art with the object of providing a flow measuring device with which an inline measurement can be realized and which reduces the aforementioned disadvantages of the prior art.

The present disclosure achieves this object by providing a third module.

The third module according to the present disclosure for a modular flow measuring device having a flow channel comprises:
a measuring chamber with a cuvette holder for arranging a cuvette in the measuring chamber,
a supply line,
a discharge line,
   wherein the supply line and the discharge line are connected to the measuring chamber in such a way that a partial section of the flow channel is formed,
a transmitting element,
a first receiving element,
a second receiving element,
   wherein the first receiving element is arranged such that a transmitted light signal path is formed between the transmitting element and the first receiving element,
   wherein the second receiving element is arranged such that a scattered light signal path is formed between the transmitting element and the second receiving element,

The above object is also achieved by providing a third module according to claim 2.

The third module according to the present disclosure for a modular flow measuring device having a flow channel comprises:
a measuring chamber with a cuvette holder for arranging a cuvette in the measuring chamber,
a supply line,
a discharge line,
   wherein the supply line and the discharge line are connected to the measuring chamber in such a way that a partial section of the flow channel is formed,
a first transmitting element,
a second transmitting element,
a third receiving element,
   wherein the third receiving element being arranged such that a transmitted light signal path is formed between the first transmitting element and the third receiving element,
   wherein the third receiving element is arranged such that a scattered light signal path is formed between the second transmitting element and the third receiving element.

The above object is also achieved by providing a modular flow measuring device according to claim 5.

The modular flow measuring device according to the present disclosure comprises:
a controller,
a third module with an optical sensor connected to the controller,
a fourth module with an amperometric sensor connected to the controller,
   wherein a flow channel fluidically sequentially connects the third module and the fourth module to one another, wherein the optical sensor has a measuring chamber, wherein the optical sensor has a cuvette holder, such that a cuvette for calibrating the optical sensor can be arranged in the measuring chamber.

The flow measuring device comprises a flow channel. A measuring medium can be guided in the flow channel.

Furthermore, the flow measuring device has an optical sensor and an amperometric sensor.

The two sensors are arranged in the fitting to measure the concentration of a measuring medium guided in the flow channel. The flow measuring device preferably determines a concentration of a species dissolved in the medium.

The flow measuring device has a cuvette holder for calibrating the optical measuring device.

The flow measuring device according to the present disclosure achieves a compact arrangement of sensors which are positioned at defined intervals through the fitting. The term “fitting” should be understood here as equivalent to the flow measuring device. The fitting also provides a transfer of the measuring medium from the optical sensor to the amperometric sensor via the flow path. A time-consuming manual transfer from a first handheld device to a second handheld device is therefore eliminated and the error is further reduced.

In addition, the flow measuring device enables permanent monitoring of the concentration of a species in the measuring medium, in particular via the amperometric sensor.

Another advantage over the prior art is that exactly the same measuring medium is compared with the amperometric sensor and therefore there is no divergence between the medium in the hand measurement and that on the amperometric sensor.

A preferred application of the flow measuring device according to the present disclosure is the measurement of drinking water and/or the water quality in swimming pools.

Advantageous embodiments of the flow measuring device according to the present disclosure form the subject matter of the dependent claims.

It is advantageous if the fitting bas an inlet and an outlet, the flow channel defining a flow path between the inlet and the outlet, the amperometric sensor being arranged downstream of the optical sensor on the flow path.

It is also advantageous if the fitting has a plurality of sensor holders for the medium-tight arrangement of the optical and amperometric sensors in the fitting, the sensors being arranged interchangeably in the fitting. Defective or inaccurate sensors can be serviced or replaced as a result.

The flow measuring device can, in particular as an inline flow measuring device, have a dosing module which is arranged in the fitting, preferably in front of the optical sensor along the flow path.

In order to determine the measuring conditions or other parameters, the flow measuring device can have a pH sensor, a flow sensor, a flow indicator and/or a conductivity sensor, which is arranged in the fitting, preferably replaceably in a sensor holder of the fitting.

The fitting is of modular design and is thus adapted to the measured variable and the accuracy requirements, wherein each module of the fitting has a partial section of the flow channel and each module has a sensor holder, the inlet or the outlet. If a module is considered on its own, it obviously does not have a partial section, but rather the flow channel.

In a compact manner, the flow measuring device according to the present disclosure can have a sensor with an optics housing as an optical sensor. Receiving and/or transmitting elements for emitting and/or receiving an optical signal for measuring the turbidity of a measuring medium and/or for determining the concentration of a species, in particular a dissolved species, in the measuring medium are arranged in the optics housing.

It is possible to provide a receiving and/or transmitting element for each signal path. However, a particularly compact design can be achieved if two or more signal paths are formed either by a single transmitting element or alternatively by a single receiving element.

Furthermore, the optical sensor has a measuring chamber in which a measuring medium can be arranged, e.g., in a cuvette. A cuvette holder within the measuring chamber is designed as a holding device, e.g., spring arms for clamping a cuvette.

The aforementioned receiving and/or transmitting elements are aligned or arranged around the measuring chamber in such a way that a scattered light signal path is provided for measuring the turbidity of a measuring medium and a transmitted light signal path is provided for determining the concentration of a species in the measuring medium.

The aforementioned signal paths run through the measuring chamber and are simultaneously guided through the measuring medium when passing through the measuring chamber.

In this case, the transmitted light signal path runs in a straight line, while the scattered light signal path is always designed as a deflected path. Preferably, the deflection and thus the arrangement or alignment of the receiving element relative to the transmitting element is 90°. This corresponds to so-called 90° scattering.

A measurement according to forward scattering or backward scattering can also be realized, in order to extend the measuring range of the turbidity sensor. An additional transmitting or receiving element can be provided for this purpose. A typical angle relative to the preferably single corresponding receiving or transmitting element for forward or backward scattering can be 45° or 135°. The extension of the measuring range results from the fact that increased backward scattering occurs at particularly high turbidity levels, and increased forward scattering occurs at very low turbidity levels with small particle sizes.

The combination of turbidity and concentration measurements in a single sensor reduces the measuring distance between the two measurements. The turbidity measurement can preferably and advantageously be used to compensate for measurement inaccuracies in the concentration measurement and/or to indicate an error tolerance of the concentration measurement.

The optical sensor can advantageously have only one transmitting element and two or more receiving elements, wherein the transmitting element is designed to emit light signals with two or more different wavelengths. A first of the wavelengths, e.g., a wavelength in the infrared range, e.g., at 520 nm, can be used to measure turbidity. A second of the wavelengths, e.g., a wavelength in the vis range, e.g., at 860 nm, can be used to measure concentration. In this case, “vis” is the commonly used term for a wavelength in the visible light range.

Overall, the existing cuvette holder allows for on-site calibration or in-place calibration to be carried out for both measurements. This avoids measurement uncertainties while maintaining a compact design of the measuring device,

Further advantageously and as an alternative to the aforementioned variant, the optical sensor can have only one receiving element and two or more transmitting elements, the receiving element being designed to receive light signals with two or more different wavelengths. Both variants enable a compact sensor design for carrying out two different measuring methods. Here, too, the receiving module can evaluate both a first wavelength at 520 nm and a second wavelength at 860 nm.

The receiving element at the end of the scattered light signal path can advantageously be designed to receive an infrared light signal and it can simultaneously be designed to receive a vis light signal.

Furthermore, the transmitting element at the beginning of the scattered light signal path is designed to emit an infrared light signal, and the transmitting element at the end of the transmitted light signal path is designed to emit a vis light signal,

The measuring chamber is designed to allow the measuring medium to flow therethrough. In addition, the measuring chamber is suitable for receiving a cuvette that can be filled with a calibration medium, or a sealed cuvette filled with, for example, fromazine,

One or more optical prisms, preferably at least three optical prisms, can be arranged along the transmitted light signal path and the measuring chamber between the transmitting element and the receiving element.

The transmitting element(s) can advantageously be designed as one or more diodes, preferably as LEDs.

One or more of the receiving elements can advantageously be formed on the transmitted light signal path as a photometric detector and/or colorimetric comparator.

Furthermore, a measuring device which comprises the optical sensor according to the present disclosure, as well as a fluid connection between the sampling location and the optical sensor for supplying a measuring medium, as well as a dosing device for adding a reagent for the colorimetric and/or photometric measurement, can also be provided. Due to the design according to the present disclosure, the measuring device can carry out a concentration measurement with a lower measurement error while taking the turbidity measurement into account. Particularly preferably, a measurement error for the concentration measurement can be output based on the turbidity measurement. Alternatively or additionally, the turbidity measurement can be taken into account when determining the concentration measurement, e.g., when compensating for a sensor drift or the like.

Furthermore, according to the present disclosure, one of the receiving elements of the optical sensor on the transmitted light signal path is designed as a photometric detector and/or colorimetric comparator.

The aforementioned object is also achieved by a method for its operation having the features of claim 14.

The method according to the present disclosure for operating the aforementioned flow measuring device according to the present disclosure comprises:
A calibration, preferably on-site calibration, of the optical sensor by inserting a cuvette with a calibration medium into the cuvette holder,
B concentration measurement of a measuring medium, conducted through the flow channel, by the optical sensor, and
C comparison and/or calibration of an amperometric concentration measurement of a measuring medium conducted through the flow channel, based on the measured values determined by the amperometric sensor with the measured values of the optical sensor.

The individual steps A-C can have further sub-steps.

It is advantageous if the turbidity of the measuring medium is also measured, preferably by the optical sensor. The sensors can also be used among themselves, inter alia for plausibility checks as part of a redundancy measurement in the event of sensor drift or jumps in measured values.

Furthermore, according to the present disclosure, the measured values determined by turbidity measurement can be compared with a target value and, if the target value is exceeded, a warning can be displayed with regard to the measurement performance of the concentration measurement.

Alternatively or additionally, for example in the case of high turbidity values, a control device can stop the flow of measuring medium into the fitting if the target value is exceeded, or switch it off completely to protect the sensors.

Alternatively or additionally, a cleaning mode for cleaning the flow channel of the fitting can be initiated based on a turbidity measurement value or from a temporal sequence of turbidity measurements.

In addition, the amperometric sensor can perform a permanent measurement and the optical sensor can perform a colorimetric and/or photometric measurement at irregular or preferably regular measurement intervals.

In the concentration measurement according to step B, in particular a reagent can be added to the measuring medium, via which a concentration value, preferably a DPD value, is determined and which is compared with the amperometric sensor. The dosing is preferably carried out discontinuously.

### BRIEF DESCRIPTION OF THE DRAWINGS

In the following, the subject matter of the present disclosure is explained in detail using an exemplary embodiment and with the aid of accompanying figures. In the figures:

FIG. 1 is a schematic structure of a first variant of an optical sensor for use in a flow measuring device according to the present disclosure;

FIG. 2 is a schematic structure of a second variant of an optical sensor for use in the flow measuring device;

FIG. 3 is a cross-sectional view of a corresponding optical sensor in the optical plane;

FIG. 4 is a vertical sectional view as a partial view of the optical sensor from FIG. 3; and

FIG. 5a is a front view of a flow measuring device according to the present disclosure with the aforementioned optical sensor;

FIG. 5b is a front view of the flow measuring device of FIG. 5a with further parts of the flow measuring device, and

FIG. 6 is a longitudinal section through an optical sensor from FIG. 3 and FIG. 4.

### DETAILED DESCRIPTION

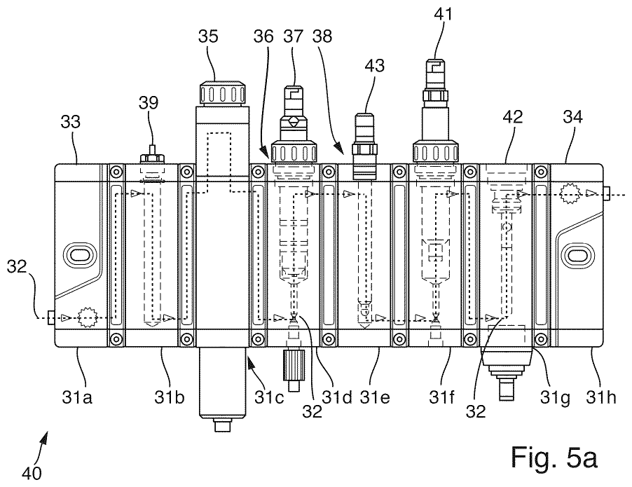

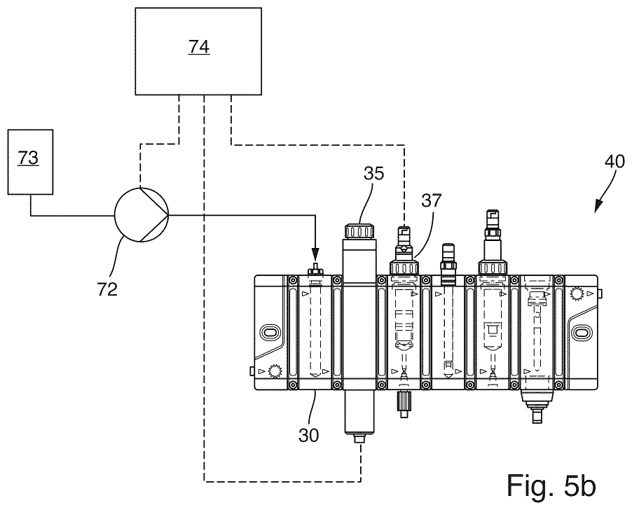

FIG. 5a and FIG. 5b each show the structure of a modular flow measuring device 40 according to the present disclosure comprising a plurality of modules 31a-31h, which are connected to one another in a medium-tight manner. Each of the modules 31a-31h has a flow channel portion, the flow channel portions resulting, in the assembled state, in a flow channel 32. The flow channel 32 is shown in FIG. 5a as a dotted line which passes through the various modules 31a-31h. The arrows shown as triangles indicate the position of an inlet and outlet of the relevant module. At the end, the fitting 30 has an inlet as an inlet module 33 with a process connection for supplying a measuring medium, preferably a liquid, and an outlet as an outlet module 34 with a process connection for discharging the measuring medium. Inlet and outlet modules 33, 34 define a flow path with a flow direction. This is highlighted in FIG. 5a with an arrow along the flow channel 32.

Furthermore, the flow measuring device 40 has an optical sensor 35 for determining the concentration of a species contained in the measuring medium, which is arranged in a sensor holder 36 of a module of the flow measuring device 40. The pH as the negative decimal logarithm of the concentration of hydronium ions in a solution is also a species mentioned above and can be used as a litmus test, for example.

The optical sensor 35 can determine the concentration by colorimetric and/or photometric measurement. The colorimetric measurement records a color spectrum of a color reagent in the measuring medium by comparing it with a reference reagent using a comparator. In contrast, the photometer does not compare a color spectrum, but only discrete wavelengths and their absorption or transmission through the measuring medium. Both occur in a transmitted light signal path through the measuring medium.

Furthermore, the flow measuring device 40 has an amperometric sensor 37, which is arranged in a sensor holder 38 of a module of the fitting 30. Amperometric sensors, e.g., for determining the concentration of disinfectant in water, have been sold by the applicant for many decades. It enables permanent measurement or monitoring of the measuring medium, in particular in the aforementioned fitting 30.

However, the amperometric measured values determined must be traced back to the measured values of an optical measurement. This allows the amperometric sensor to be compared with the optical sensor from time to time and/or calibrated using the optical sensor. Therefore, the amperometric sensor 37 is ideally arranged downstream of the optical sensor 35 on the flow path, but can also be installed upstream.

In an inline measurement, a dye and/or a color indicator must in general be added to the measuring medium for each measurement to color the species. This is done by a dosing module 39, which is ideally arranged in front of the optical sensor 35, i.e., between the optical sensor 35 and the inlet on the flow path.

The two concentration measurement methods, optical and amperometric, are in many cases dependent on the measurement conditions and on the measuring medium in which the species is dissolved. To determine these measuring conditions, the fitting optionally has one or more additional sensors. These comprise a pH sensor 43, a conductivity sensor 41, a flow sensor and/or a flow indicator 42, which are preferably arranged in a sensor holder, particularly preferably in a relevant module 31e-31g of the fitting 30.

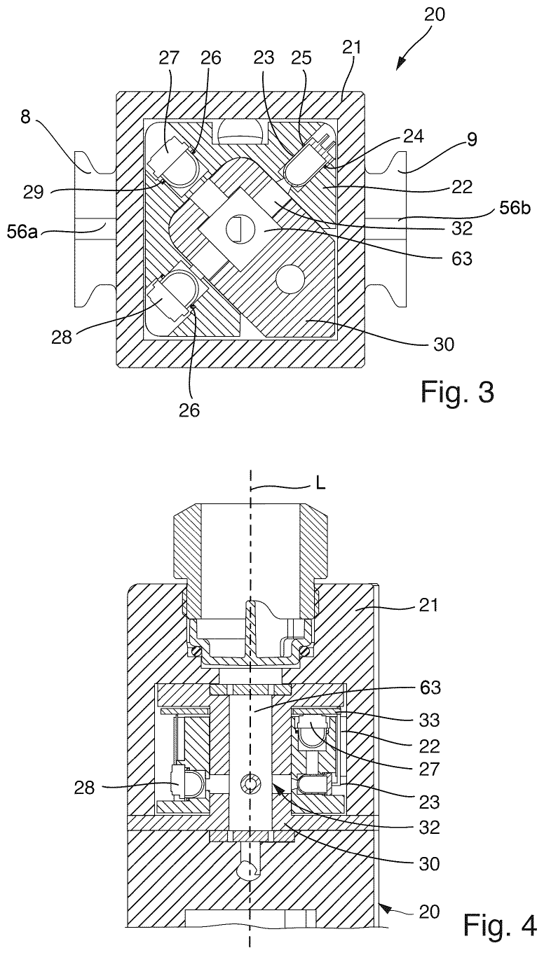

The modules 31a-31h are connected to one another in a medium-tight manner and are preferably arranged so that they can be separated from one another by releasing a mechanical connection, e.g., a screw connection. As can be seen in FIG. 3, for this purpose the modules have a first flange 8 and a second flange 9. The modularity of the fitting allows the fitting to be expanded with additional measuring sensors, which, for example, enable a more precise determination of the measuring conditions and/or the measuring medium. Still other concentration determinations are only slightly sensitive to interference, In this case, the fitting can also be composed of fewer modules.

Another important parameter is the turbidity measurement. The present disclosure comprises a particularly compact arrangement of an optical sensor which combines a cuvette holder, a turbidity measurement and a calorimetric and/or photometric measurement in one sensor.

The use of a cuvette holder, especially in an optical sensor in the fitting, enables inline calibration of the optical sensor without removing the optical sensor 35 from the fitting 30.

The concentration measurement of the optical sensor can be used to calibrate or compare the amperometric sensor.

The additional optional determination of the turbidity content can be used to detect deposits such as red algae or black algae, wastewater deposits and the like. Stronger turbidity can also damage the measuring membrane of the amperometric sensor or lead to inaccurate measurements. Here too, the flow measuring device according to the present disclosure can issue a warning signal for the risk of an inaccurate measurement. Alternatively or additionally, the supply to the fitting 30 can be shut off by a control device to prevent further damage.

Alternatively or additionally, the turbidity measurement can be used to initiate cleaning, e.g., a cleaning program, or set a cleaning interval. The turbidity measurement can be used to indicate a measurement uncertainty, for example in the concentration measurement of process water. In this case, high turbidities are associated with a higher measurement uncertainty than low turbidities.

The combination of both measuring methods allows reliable inline measurement, thus reducing manual sources of error. Furthermore, the amperometric sensor enables continuous and precise concentration determination with low dye consumption.

Self-evidently, the turbidity can also be specified simply as an important quality parameter when examining the measuring medium.

The flow measuring device according to the present disclosure has the particular advantage of an inline measurement without removing the sensor, in particular the optical sensor, from the fitting, for its calibration.

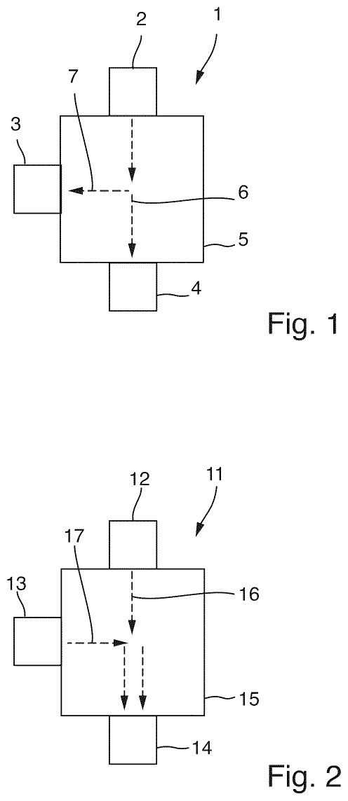

FIG. 1-4 show the structure of an optical sensor for realizing a concentration measurement and a turbidity measurement, the sensor being characterized by its compact design.

FIG. 1 shows the schematic structure of the optical sensor 1 for determining the concentration and for measuring the turbidity in a measuring medium and for use as part of the flow measuring device 40 of FIG. 5a or FIG. 5b.

Sensors for measuring concentration based on the optical principle are known in principle. The concentration measurement of one or more species in the measuring medium can be carried out in particular by photometric measurement and/or by colorimetric measurement.

A typical example of the measuring principle is the so-called DPD measurement, which is used to determine the chlorine and/or perchlorate content in water.

DPD measuring devices are used optionally as handheld devices for determining chlorine levels in pools and swimming pools, where the chlorine content must sometimes be determined several times a day due to legal requirements. For this measurement, a color chemical must be added to the water in a cuvette, which is usually added to the measuring medium in powder or tablet form. The cuvette is then placed in a cuvette holder within the handheld device, the cuvette being positioned in a beam path in the handheld device.

For larger swimming pools and amusement parks, there are also so-called online systems for the continuous supply and removal of measuring medium. In these online systems, the color chemical is preferably added to the sample at measuring intervals, the measuring medium pretreated in this way then being fed into the beam path. In terms of dimensions, such systems cannot be compared to a handheld device. The consumption of chemicals is comparatively high, but the measurement results are available quickly and are less error-prone due to more consistent measurement conditions.

Both colorimetric and photometric measurements can be used to optically determine the concentration of a species in the optical sensor.

Colorimetric measurement involves an optical comparison of the color and/or color depth between a test sample of the medium and a reference, e.g., a color standard. This can be, for example, a color screen. The test sample of the measuring medium can be prepared by removing it and transferring it into a cuvette. Online measurement through a flow cell, e.g., in a bypass method, is also conceivable. The sample and the reference are compared by a comparator.

The sensor consists of two or more transmitting elements and only one receiving element, or alternatively of only one transmitting element and two or more receiving elements. Both variants are shown schematically in FIGS. 1 and 2.

A light source, e.g., a light source, possibly in combination with a slit diaphragm and a so-called monochromator, can be used as a transmitting element. A significant simplification and at the same time miniaturization is made possible, for example, by the use of a photodiode and/or an LED as a light source. LEDs can produce monochromatic light, i.e., light in a specific wavelength. LEDs that can produce monochromatic light with multiple wavelengths, which is particularly advantageous for the present application since the turbidity measurement is carried out at a different wavelength than the concentration measurement, are known.

The monochromatic light can then be directed onto the measuring cuvette containing the water sample. Alternatively, a flow cell can also be used. The water sample was previously colored, the intensity of the color depending on the concentration of the species to be identified. In the case of the flow cell, a mixing chamber is fluidically connected upstream of the measuring chamber, in which mixing chamber the sample is added and mixed. The dye absorbs light at a specific wavelength, the absorption depending on the concentration of the species to be determined in the measuring medium. The light conducted through the measuring medium can be conducted through an interference filter so that the light is received at a defined wavelength. Due to their design, slight fluctuations in wavelength are normal for many light sources over the course of their life cycle, Finally, the light is directed to a detector. For example, photodiodes can be used for this purpose, which photodiodes convert the incoming light into an electrical signal. Detector and interference filter can be part of a receiving element. The receiving element may optionally also include a photomultiplier for amplifying the received signal. The concentration of the species can typically be determined according to the Lambert-Beer law.

The structure of both a colorimeter and a photometer are known. While the comparator is used as the receiving element in the colorimeter, in the photometer it is at least the aforementioned detector, possibly in combination with other components, such as the interference filter.

In addition to the actual optical measurement, depending on which species is to be determined, additional measured variables can be determined or additional reagents can be added to the measuring medium to adjust the measuring conditions. For this purpose, a flow measuring device can have additional sensors in addition to the optical sensor. For example, the DIN-standardized DPD method for determining chlorine can also determine the pH. In addition to the DPD reagent—i.e., N,N-diethyl-p-phenylenediamine—a pH indicator, such as phenol red, can be added. Buffer reagents are also used, so that the chlorine content has no influence on the pH measurement. In the chlorine measurement, the optimal pH is set.

The procedure of a colorimetric and/or photometric measurement is known per se, To put it simply, in the measuring chamber, i.e., the cuvette or the flow cell, a zero comparison is first made with the measuring medium without dye and then the dye is added. The measurement then takes place.

A further measurement that can be carried out with the optical sensor is the turbidity measurement. The formazin standard is typically used for the turbidity measurement,

An infrared light source can be used as a light source according to ISO 7027:1999. In this case, the infrared measurement is not influenced by the color of the medium.

Alternatively or additionally, a white light source in the visible range according to US-EPA 180.1 can be used as the light source.

Scattered light measurement and transmitted light measurement are known methods for measuring turbidity. In the case of the present optical sensor, scattered light measurement is used.

This measurement distinguishes between forward scattering, backward scattering and 90° scattering. The standard procedure according to 7027 and US-EPA 180.1 is the 90° measurement.

However, in very turbid media, backscattering can also be determined at an angle between 90 and 180°, preferably 100-170°, for example at 135°. An additional receiver can be provided for this purpose.

In contrast to the scattered light measurement, transmitted light measurement records the light that passes through. This definition for the transmitted light measurement is also applicable to the colorimetric and/or photometric measurement.

FIG. 1 shows an optical sensor 1 with a single transmitting element 2 and two receiving elements 3 and 4.

The optical sensor 1 also has a measuring chamber 5 for arranging a cuvette or for the flow of a measuring medium. The measuring chamber can, for example, comprise a holder for a cuvette.

The arrangement of the transmitting element 2 and a first receiving element 4 around the measuring chamber 5 is such that a transmitted light signal path 6 is provided between the transmitting element 2 and the first receiving element 4. The first receiving element 4 is also referred to below as the transmitted light receiving element.

The arrangement of the transmitting element 2 and a second receiving element 3 around the measuring chamber 5 is such that a scattered light signal path 7 is provided between the transmitting element 2 and the second receiving element 3.

The second receiving element 3 is also referred to below as the scattered light receiving element.

The transmitting element 2 is preferably equipped to emit light signals with two different wavelengths. The transmitting element can be designed, for example, as a photodiode and/or LED. For example, the LED can be designed as a photodiode.

In FIG. 2, the optical sensor 11 has two transmitting elements 12 and 13 and a single third receiving element 14.

The optical sensor 1 bas, analogously to FIG. 1, a measuring chamber 15 for arranging a measuring medium,

The arrangement of a first transmitting element 12 and the third receiving element 14 around the measuring chamber 15 is such that a transmitted light signal path 16 is provided between the first transmitting element 12 and the third receiving element 14.

The arrangement of a second transmitting element 13 and the third receiving element 14 around the measuring chamber 15 is such that a scattered light signal path 17 is provided between the second transmitting element 13 and the third receiving element 14.

The third receiving element 14 is equipped for evaluation, preferably for simultaneous evaluation, of two wavelengths.

FIG. 3 shows a more detailed structure of an optical sensor 20 in the variant of FIG. 1.

The optical sensor 20 has an optics housing 21. An optics carrier 22, e.g., in the form of a plastics body and/or a circuit board, is arranged within the optics housing. The optics carrier 22 has an enclosure 25 for a light source 23 of a transmitting element. In this case, the light source 23 can be mounted interchangeably within the optics carrier 22. The enclosure 25 preferably has a stop surface 24 so that the light source 23 is at a defined distance from the measuring medium.

Furthermore, the optics carrier 22 has further enclosures 26 for a receiving module 27, 28, each as part of a first and a second receiving element. The receiving module can be designed as a diode. These enclosures 27, 28 also have stop surfaces 29 for forming a defined distance between the respective receiving modules 27, 28 and the measuring medium.

An optical flow cell 30 can be arranged between one or more of the receiving modules 27, 28 and the light source 23. This, for example, is designed like the cuvette 63, only without a base, i.e., as a tube.

The receiving module 27 is part of the transmitted light receiving element, and the receiving module 28 is part of the scattered light receiving element.

Optical prisms can be arranged between the receiving module 27 of the transmitted light receiving element and the light source 23 of the flow cell 30.

FIG. 4 shows the sensor base body with the optics housing 21, An optical circuit board 33 can be arranged in each case at the end of the light source 23 and each of the receiving modules 27, 28 for signal transmission, signal tapping and/or, if necessary, for signal processing of the received light signals.

In FIG. 4, the sensor base body is elongate. It has a longitudinal axis. The elongate measuring chamber 5 runs along this longitudinal axis L and is preferably provided by the optical flow cell 30.

In addition, a return channel runs radially from the measuring chamber 5 through the optics housing 21 to the outside of the optical sensor 20.

The sensor 1, 20 described above combines a plurality of measuring principles, usually used separately in a plurality of sensors, in one sensor. The sensor design is correspondingly compact. At the same time, the separate replacement of the light source and the receiver creates the possibility of replacing individual components of the sensor in a simple manner as soon as the first fluctuations, e.g., due to age, occur, so that the optical sensor 1, 20 can be repaired without any problems using simple means.

Furthermore, the sensor 1, 20 includes a control and/or evaluation unit (not shown in detail), which can be arranged outside on the sensor, for example as a transmitter head, and which allows the wavelengths to be set and the operating modes “turbidity measurement on/off” or “transmitted light measurement on/off” to be switched on and/or off separately, or even the simultaneous operation of both operating modes.

At the same time, in the case of a light source with a wavelength that can be set in a variable manner, a desired wavelength and/or a wavelength optimized for the species to be measured can be set by the control and/or evaluation unit.

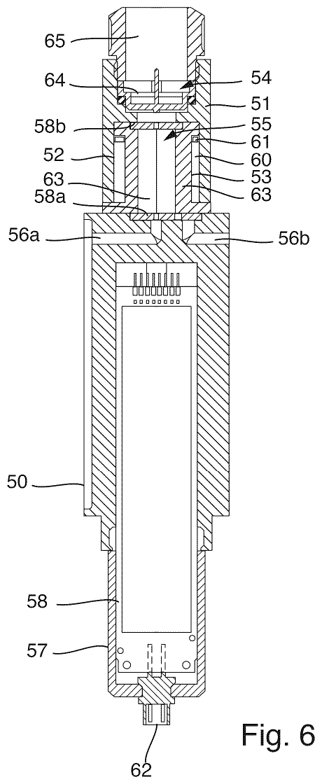

FIG. 6 is a longitudinal section through an optical sensor 50 for arrangement in the flow measuring device 40. This has an optics housing 51 and a measuring chamber 55 formed therein, around which at least one transmitting element 52 and one receiving element 53 are arranged. A cuvette holder 54 is arranged inside the measuring chamber 55. The measuring chamber 55 can be flooded with the measuring medium via a supply line 56a arranged in the optics housing 51, which medium can be drained via a discharge line 56b arranged in the optics housing. In this case, the measuring medium is introduced from a base surface of the measuring chamber 55 into said measuring chamber. In this case, the supply line 56a and the discharge line 56b extend perpendicularly to the base surface in some regions, in order to safely remove air bubbles with the flow. The seals and channels mentioned or shown in the context of FIG. 6 are designed in such a way that the formation of condensate is advantageously prevented.

An electronics housing 57 is arranged below the measuring chamber. The optics housing 51 and the electronics housing 57 can also be connected in one piece as housing segments to form an overall housing. The electronics housing has an interior in which a sensor electronics 58 is arranged. At the end, the electronics housing has an electrical sensor connection 62. The measuring chamber 55 can have a resilient seal on the base and/or ceiling side, in particular a flat seal 58a and 58b, onto which a cuvette 63 positioned in the cuvette holder 54 can be placed and which forms part of the cuvette holder, e.g., in the form of a holding device for clamping the cuvette at the end, The cuvette holder 54 can enable a clamping hold of the cuvette 63. The measuring chamber 55 is axially closed by a blind plug 64 and a pressure screw 65, but other closure variants are also conceivable.

As in FIG. 3-4, the transmitting and/or receiving element 52 and 53 can be arranged in an optics carrier 60, e.g., a potting compound. Furthermore, the transmitting and/or receiving module 52 and 53 is connected to an optics electronics 61, e.g., a circuit board.

The cuvette 63 is designed to be closable or closed so that, for example, calibration medium cannot escape. It is self-evident that the measuring chamber 55 must be emptied before inserting the cuvette 63 into the cuvette holder 54.

The optical sensor can be calibrated as follows.
a) interrupting the flow of measuring medium through the flow channel of the fitting, in particular through a flow channel segment of the optical sensor;
b) opening the optical sensor and making it accessible and, if necessary, emptying the measuring chamber;
c) optional cleaning of the measuring chamber;
d) inserting the cuvette in the measuring chamber into the cuvette holder;
e) controlling the measuring function to initiate a calibration, e.g., by a transmitter;
f) removing the cuvette from the measuring chamber of the optical sensor after calibration is complete;
g) closing the optical sensor and/or the entire fitting;
h) setting a flow rate of a measuring medium.

After the previously described calibration of the optical sensor by inserting a cuvette with a calibration medium into the cuvette holder, a concentration measurement of a measuring medium conducted through the flow channel can then be carried out by the optical sensor.

Finally, a comparison and/or calibration of an amperometric concentration measurement of a measuring medium conducted through the flow channel can be carried out using the measured values determined by the amperometric sensor with the measured values of the optical sensor,

The reference measurement or the cleaning is automated via a pump 72, which supplies a corresponding reagent from a storage container 73 to the dosing module via a connection on the fitting, or pumps it into the supply line (see FIG. 5b).

In this case, the pump can be adjusted by a programmable logic controller 74 or a PLC, or by a control and/or evaluation unit or by a transmitter. By means of a transmitter controller, the pump 72 can be activated as required, at time intervals or based on measured values from the optical sensor. The control of a cleaning mode based on measured values can also be set in the PLC or in an autonomous cleaning controller, according to a degree of contamination (since an increasing degree of contamination leads to a weakening of the light), so that cleaning is then activated based on a defined limit value.

1. A third module for a modular flow measuring device with a flow channel, comprising:
a measuring chamber with a cuvette holder for arranging a cuvette in the measuring chamber;
a supply line;
a discharge line, wherein the supply line and the discharge line are connected to the measuring chamber in such a way that a partial section of the flow channel is formed;
a transmitting element;
a first receiving element; and
a second receiving element,
wherein the first receiving element is arranged such that a transmitted light signal path is formed between the transmitting element and the first receiving element, and
wherein the second receiving element is arranged such that a scattered light signal path is formed between the transmitting element and the second receiving element.

2. A third module for a modular flow measuring device with a flow channel, comprising:
a measuring chamber with a cuvette holder for arranging a cuvette in the measuring chamber;
a supply line;
a discharge line, wherein the supply line and the discharge line are connected to the measuring chamber in such a way that a partial section of the flow channel is formed;
a first transmitting element;
a second transmitting element; and
a third receiving element,
wherein the third receiving element is arranged such that a transmitted light signal path is formed between the first transmitting element and the third receiving element, and
wherein the third receiving element is arranged such that a scattered light signal path is formed between the second transmitting element and the third receiving element.

3. The third module according to claim 1, wherein the third module has a first flange and a second flange, wherein the supply line opens into the first flange and the discharge line opens into the second flange, wherein the first flange and the second flange are suitable for connecting the third module to a further module of the modular flow measuring device.

4. The third module according to claim 1, wherein the cuvette holder is closable by a blind plug.

5. A modular flow measuring device, comprising:
a controller;
a third module with an optical sensor connected to the controller; and
a fourth module with an amperometric sensor connected to the controller,
wherein a flow channel fluidically sequentially connects the third module and the fourth module to one another, and
wherein the optical sensor has a measuring chamber and further has a cuvette holder such that a cuvette for calibrating the optical sensor can be arranged in the measuring chamber.

6. The modular flow measuring device according to claim 5, further comprising:
a dosing module which is arranged in front of the optical sensor along the flow path.

7. The modular flow measuring device according to claim 6, further comprising:
a pH sensor replaceable in a fifth module;
a flow indicator in a seventh module; and/or
a conductivity sensor replaceable in a sixth module.

8. The modular flow measuring device according to claim 7,
wherein each module has a partial section of the flow channel, and wherein each module has a sensor holder, the inlet or the outlet.

9. The modular flow measuring device according to claim 5, wherein the optical sensor has an optical housing in which receiving and/or transmitting elements are arranged for emitting and/or receiving an optical signal, in each case, for measuring the turbidity of a measuring medium and/or for determining the concentration of a species in the measuring medium, and which has a measuring space in which a measuring medium can be arranged, wherein the receiving and/or transmitting elements are aligned around the measuring space such that a scattered light signal path for measuring the turbidity of a measuring medium and a transmitted light signal path for determining the concentration of a species in the measuring medium are formed.

10. The modular flow measuring device according to claim 5, wherein the optical sensor has only one transmitting element and two or more receiving elements, wherein the transmitting element is designed to emit light signals with two or more different wavelengths, or
the optical sensor has only a third receiving element and two or more transmitting elements, wherein the third receiving element is designed to receive light signals with two or more different wavelengths.

11. The modular flow measuring device according to claim 9, wherein the receiving element at the end of the scattered light signal path is designed to receive an infrared light signal, and the receiving element at the end of the transmitted light signal path is designed to receive a visible light signal, or
the transmitting element at the beginning of the scattered light signal path is designed to emit an infrared light signal, and the transmitting element at the end of the transmitted light signal path is designed to emit a visible light signal.

12. The modular flow measuring device according to claim 5, wherein the measuring chamber has the cuvette holder and has a cuvette that can be filled with measuring medium and/or a flow cell that is designed to allow permanent flow therethrough.

13. The modular flow measuring device according to claim 9, wherein one of the receiving elements of the optical sensor on the transmitted light signal path is designed as a photometric detector and/or colorimetric comparator.

14. A method for operating a modular flow measuring device, the method comprising:
providing the modular flow measuring device, including,
a controller;
a third module with an optical sensor connected to the controller; and
a fourth module with an amperometric sensor connected to the controller,
wherein a flow channel fluidically sequentially connects the third module and the fourth module to one another, and
wherein the optical sensor has a measuring chamber and further has a cuvette holder such that a cuvette for calibrating the optical sensor can be arranged in the measuring chamber;
calibrating the optical sensor by inserting a cuvette with a calibration medium into the cuvette holder;
measuring a concentration of a measuring medium, conducted through the flow channel, by the optical sensor; and
comparing and/or calibrating of an amperometric concentration measurement of a measuring medium conducted through the flow channel based on measured values determined by the amperometric sensor and the measured values of the optical sensor.

15. The method according to claim 14, further comprising:
measuring a turbidity of the measuring medium.

16. The method according to claim 15, further comprising:
comparing the measured values determined by turbidity measurement with a target value and that, if the target value is exceeded, displaying a warning with regard to a measurement performance of the concentration measurement, and/or
stopping an inflow of measuring medium into the modular flow measuring device if the target value is exceeded, and/or
initiating a cleaning mode for cleaning the flow channel of the modular flow measuring device.

17. The method according to claim 14, further comprising:
performing a permanent measurement with the amperometric sensor; and
performing a colorimetric and/or photometric measurement with the optical sensor at irregular or regular measurement intervals.

18. The method according to claim 14, wherein a reagent is dosed during the concentration measurement step via which reagent a concentration value is determined which is compared with the amperometric sensor.
